(19) United States
(12) Patent Application Publication
(10) Pub. No.: US 2011/0217363 A1
(43) Pub. Date: Sep. 8, 2011

(54) TWO-STEP TARGETED TUMOR THERAPY WITH PRODRUG ENCAPSULATED IN NANOCARRIER

(75) Inventor: Jun Chen, San Jose, CA (US)

(73) Assignee: BIONANOX, San Jose, CA (US)

(21) Appl. No.: 13/040,509

(22) Filed: Mar. 4, 2011

Related U.S. Application Data
(60) Provisional application No. 61/311,287, filed on Mar. 5, 2010.

(51) Int. Cl. A61K 38/43; A61K 9/00; A61K 9/127; A61K 39/395; A61P 35/00

(52) U.S. Cl. 424/450; 424/94.1; 424/400; 424/178.1; 424/94.3

(57) ABSTRACT

A two-step targeted tumor therapeutic method is described comprising nanocarrier with prodrug encapsulated thereto and prodrug activating enzyme. The enzyme is either encapsulated in nanocarrier, or not encapsulated, or synthesized in the tumor. With the method, the prodrug released from the nanocarrier is activated by the enzyme mainly in the tumor.

### CROSS-REFERENCE TO RELATED APPLICATION

This application claims the benefit of PPA Ser. No. 61/311,287, filed Mar. 5, 2010, which is incorporated by reference.

### BACKGROUND OF THE INVENTION

For optimal cancer chemotherapy, the drug concentration must be maintained on an effective level at the target site for a sufficient duration, with minimal dose accumulation at non-target sites. However, current chemotherapy is generally not selective and, as a result, it exerts serious side effects. Even for the molecularly targeted therapeutics, which is the major interest of current biomedical research and commercial effort, off-target effects and/or shared target between tumor and normal tissues are still the major challenges to overcome. Additionally, the current chemotherapeutic drug usually cannot maintain its effective level for a sufficient period of time, which leads to less optimal efficacy and drug resistance.

New strategies for targeted cancer drug delivery have been explored in recent decades and significant progresses have been made, among which, notably, are immunoconjugate, antibody-directed enzyme prodrug therapy (ADEPT) and nanomedicine. However, major obstacles still exist for these technologies to realize their potentials.

lmmnunoconjugate:

Coupling a therapeutic agent to a carrier molecule (e.g. antibody) with specificity to a defined target population of cells is an attractive strategy to achieve site-specific drug delivery. Considerable success has been made on this drug delivery strategy, especially for immunoconjugates (antibody-drug conjugate, antibody-toxin conjugate, and antibody-radionuclide conjugate)(Alley et al. 2010; Teicher 2009; Gerber et al. 2009a; Gerber et al. 2009b; Low and Kularatne 2009). So far, Bexxar® and Zevalin®, anti-CD20-radio-immunoconjugates, are in the market and many more are in different stages of development. However there are many limitations with this delivery strategy, including: i) Low drug-antibody ratio results in low drug concentration; ii) No tumor specific surface marker has been found, and current targeting agent still binds to normal cell or tissue in some degree; iii) Poor penetration of the conjugates to the deeper targets because of its size; iv) Some cancer cells in the tumor do not express the target, and will survive and expand; v) Off-target uptake by normal tissues, especially for liver.

Antibody-Directed Enzyme Prodrug Therapy (ADEPT):

An enzyme (or other activator) is conjugated to a targeting antibody (or other targeting moiety). After the conjugate localizes in the target and cleared from the circulation, a prodrug is delivered and converted to active drug by the enzyme only in the targeted area. This site-specific activation of prodrug results in a high local concentration of the active chemotherapeutic agent in tumor with minimal exposure to healthy tissues(Singh et al. 2008; Kratz et al. 2008). Some ADEPT systems have progressed to clinical trials (Bagshawe 2009). However, despite its promising potential in targeted drug delivery, ADEPT has significant obstacles to overcome before its success in clinic. It requires: i) No or little endogenous substrate to compete for the enzyme; otherwise, the endogenous substrate will decrease the enzymatic activity toward prodrug, and adverse consequence may occur because of depleted endogenous substrate; ii) No or little endogenous enzyme outside the targeted tumor; iii) No immunogenicity for the enzyme, antibody, or antibody-enzyme conjugate; iv) The antibody-enzyme conjugate needs to be cleared from circulation before delivery of prodrug, which results in limited duration of effective enzyme concentration in the target; v) Most prodrugs are small molecules and have short half-life, which results in short exposure of active drug to tumor.

A similar strategy to ADAPT, Gene-Directed Enzyme Prodrug Therapy (GDAPT) (Altaner 2008; Houston 2007), has also been actively investigated. Among many other obstacles, efficient targeted gene delivery is a major challenge as it is for all gene therapies so far.

Nanomedicine:

Nanomedicine uses a different strategy for targeted tumor therapy (Torchilin 2010; Mikhail and Allen 2009). The unique structural features of many solid tumors (hypervasculature, defective vascular architecture, extensive production of vascular permeability enhancing factors, and impaired lymphatic drainage) lead to relatively selective extravasation and retention inside the interstitial space of tumor for long circulating nano-sized molecules and nanocarriers, ranging from 10 to 500 nm in size. This phenomenon, enhanced permeability and retention (EPR), is essentially the working principle of most clinically viable targeting strategies based on nanocarriers and macromolecules such as PEGylated enzyme (Maeda et al. 2009; Sawa et al. 2000; Fang et al. 2002; Greish 2007a). Various nanocarriers, such as polymeric micelles, polymeric nanoparticles, liposomes, polymersomes, nanospheres, nanocapsules, dendrimers, proteins, cell ghosts, inorganic/metallic nanoparticles, and magnetic and bacterial nanoparticles are widely explored for experimental and even clinical delivery of therapeutic and diagnostic agents (Alexis et al. 2010; Matsumura and Kataoka 2009; Wang et al. 2009). PEGylated liposomal doxorubicin (Doxil, or Daelyx in Europe), which is approved for clinical use in Kaposi's sarcoma and advanced ovarian cancer, substantially diminishes the side-effects characteristic of free doxorubicin and demonstrates high efficacy in EPR-based tumor therapy (Huober et al. 2010; O'Brien 2008b).

The characteristics of nanocarrier mediated drug delivery include: i) Target tumor through EPR or EPR plus active targeting with linked targeting moiety; ii) High drug loading capacity; iii) Long circulation time and, therefore, long half-life of the drug; iv) Higher drug dosage may be allowed because of reduced side-effect; v) Deliver both hydrophilic and hydrophobic drugs.

Nanocarrier can also mediate targeted delivery of therapeutic drugs to other pathologic areas such as inflammation sites of inflammatory diseases and infarcts because of leaky vasculature associated with these disorders (Torchilin 2010).

Drug delivery through nanocarriers can, in theory, overcome the obstacles of non-selectivity, less optimal drug concentration and short exposure time, the main limitations associated with chemotherapy. However nanomedicine has its own shortcomings to overcome before realizing its full potential: i) Rapid clearance of nanocarrier by reticuloendothelial system (RES) such as liver and spleen; ii) Toxicity in RES organs because of uptake of the nanocarriers; iii) Toxicity associated with drug release in the circulation and other non-tumoral location, especially during the early period of drug administration when blood concentration of the nanocarrier is high.

Doxil, for example, still shows myelosuppression and hand-food syndrome among other side-effects, although it reduces cardiac toxicity of doxorubicin (O'Brien 2008a; Huober et al. 2010).

As an effort to improve the efficacy of macromolecule based drug delivery, Duncan group has proposed a different two-step anti-cancer approach, Polymer-Directed Enzyme Prodrug Therapy (PDEPT) (Duncan et al. 2001; Satchi-Fainaro et al. 2003). PDEPT is composed of delivery of polymeric prodrug, followed 5 h later (time for the clearance of polymeric prodrug from the circulation) by delivery of polymer-enzyme conjugate to release drug from the polymeric prodrug accumulated in the tumor. Preclinical studies showed significant decrease in tumor growth. However, the short circulation half-time for polymeric prodrug will limit the accumulation of the prodrug in the tumor, since long circulation time (at least 6 h) is found for optimal accumulation of nanocarriers in tumor (Greish 2007b). This approach has other drawbacks, including the possible activation of prodrug in lysosomes of RES and other non-tumoral cells, and less optimal accessibility of prodrug to the enzyme when both are linked to separate macromolecules.

Reticuloendothelial System (RES):

RES has been defined as a phagocytic system comprising blood monocytes and tissue macrophages localized in places such as liver, spleen, lymph nodes, bone marrow, and lung (Albert B. et al. 2002; Hume 2006). RES is ideally positioned to recognize, bind, and phagocytose foreign particles such as latex, colloidal carbon or bacteria, and senescent blood cells present in the circulation. As a result, when nanocarriers and macromolecules are administered into circulation, they are usually recognized and cleared by RES, notably by Kupffer cells and hepatocytes in liver and macrophages in spleen (Huang et al. 2010). Consequently, RES limits systemic drug delivery by nanocarrier and macromolecules, and causes toxicity and impairs the function of RES organs. Extensive efforts have been made for nanocarriers to evade RES, and the results are still far from satisfactory (Alexis et al. 2008). For example, with the use of “STEALTH®” technique, liver and spleen are still the main deposit sites of these “stealth” nanocarriers (Allen and Martin 2004; Cui et al. 2007).

The present invention explores the advantages and avoids the shortcomings of strategies of immunoconjugate, ADEPT, and nanomedicine, and establishes novel methods for sustained and targeted delivery of chemotherapeutic drugs with little or much reduced side-effects.

### SUMMARY OF THE INVENTION

This invention describes a strategy for sustained targeted delivery of active chemotherapeutic drugs to tumor. The same strategy can also be used for inflammatory diseases and potentially other indications such as infectious and parasitic diseases and atherosclerosis. In addition to the therapeutic ability, the presently disclosed and claimed inventive concepts have potential application for disease diagnosis, as they can be used to specifically pin point, target, identify, or locate the location of tumors and other pathologic lesions.

The strategy is a two-step delivery of nanocarrier with prodrug encapsulated thereto and prodrug-activating enzyme (or other activating agent). The encapsulated prodrugs are protected from activation by the enzyme until their release from the nanocarrier. The enzyme in this strategy either exists as a free entity, or is encapsulated in a nanocarrier, or is expressed at the target sites. The prodrug is non-toxic or much less toxic than the parent drug and is not activated inside lysosomes by lysosomal enzymes and acidic environment. Both the nanocarrier and activating enzyme have a long circulating half-time in the blood, and accumulate in solid tumor through EPR.

When nanocarriers are cleared in RES, they are mainly engulfed (or endocytosed) by phagocytic cells and end up in lysosomes. Because prodrug cannot be activated inside the lysosome, engulfment (or endocytosis) of the prodrug encapsulated nanocarrier will not cause damage to the cells and the tissue nearby. However, when nanocarriers are accumulated in tumor through EPR, high concentration of the prodrug will be released and accumulated in the interstitium of tumor and is activated by the enzyme also accumulated in the tumor. Similarly, the activating enzyme, either encapsulated or non-encapsulated, can be cleared by RES through engulfment. Once inside the endosome and lysosome, the enzyme will be denatured and digested, thus losing its activity. Since the nanocarrier and enzyme do not accumulate in the interstitium of RES (they either are engulfed by RES or flow through), little prodrug activation occurs in RES. However, accumulation of prodrug and enzyme occurs in the interstitium of solid tumor, and major cytotoxicity happens only in the tumor.

Although the prodrug will be released outside the tumor as the nanocarrier circulates in the circulation system, especially during the early period of high blood concentration of nanocarrier, the toxicity occurs only when certain amount of activating enzyme is nearby and the prodrug concentration is above certain level. By encapsulating the enzymes inside a nanocarrier, or expressing the enzymes in tumor, little amount of prodrug will be activated outside the tumor. For non-encapsulated enzymes, drug activation outside the tumor can be substantially reduced by avoiding simultaneous concentration peak of the nanocarrier and enzyme, which can be achieved with properly designed schedule of administration for the nanocarrier and enzyme.

A variety of optimization methods can further increase the targeted activation of prodrug and reduce the non-targeted activation. These include PEGylation of the nanocarrier and activating enzyme, adaptation of smart nanocarriers with accelerated release of the encapsulated cargo in the environment specific to tumor such as low pH, low oxygen level, and high temperature, and active targeting by attaching a tumor targeting moiety to the enzyme and nanocarrier.

PEGylation of nanocarrier and enzyme is a common strategy to increase their circulation time and reduce their endocytosis by RES, which results in enhanced accumulation of the nanocarrier and enzyme in the tumor interstitium (Osada et al. 2009; Torchilin 2010; Alexis et al. 2010; Veronese and Mero 2008; Pasut and Veronese 2009). PEGylation also inhibits endocytosis of the nanocarrier and enzyme by tumor cells and tumor stromal cells, which further increases their accumulation in the tumor interstitium. In addition, PEGylated enzyme is more stable and resistant to degradation by proteases (Roseng et al. 1992) which are highly expressed in tumor interstitium. PEGylated heterologous (foreign) enzyme can be used in the presently disclosed inventive embodiments because of no or greatly reduced antigenicity and immunogenicity achieved with PEGylation.

Tumor microenvironment responsive nanocarrier and active targeting nanocarrier have been explored intensively in recent years to enhance the therapeutic and diagnostic properties of the nanocarriers (Mikhail and Allen 2009; Kim et al. 2009; Torchilin 2009). Most solid tumors show local hypoxia and accumulation of acidic metabolites. Accelerated release of nanocarrier encapsulated cargo in response to these conditions will increase the drug concentration in the target and enhance the efficacy. Similarly, with attachment of targeting moieties for tumor cells, tumor extracellular matrix, or tumor vasculature, the nanocarrier and enzyme will accumulate more or stay longer in the tumor and, therefore, have better efficacy.

As a result, with the optimally designed embodiments of this invention, a sustained high level of active drug in the tumor with substantially decreased side effect in both RES and other non-tumoral tissues will be achieved.

### BRIEF DESCRIPTION OF THE DRAWINGS

FIG. 1. Duel nanocarrier system for targeted cancer therapy. The figure depicts the mechanism of action of targeted drug activation with duel nanocarrier system. The prodrug and prodrug-activating enzyme are encapsulated in two separate nanocarriers. When the nanocarriers reach RES (liver and spleen are chosen for exemplary purpose), some of them are phagocytosed by receptor-mediated endocytosis, and others pass through. The phagocytosed enzyme is denatured and degraded in the lysosomal system. The phagocytosed prodrug cannot be activated inside lysosomes by lysosomal enzymes and acidic environment, and thus causes no toxicity to RES organs. When both nanocarriers are accumulated in the extracellular space in tumor through the mechanism of EPR, they release enzyme and prodrug respectively. The prodrug is activated by the enzyme, and kills tumor cells.

FIG. 2. Prodrug nanocarrier and PEGylated enzyme system for targeted cancer therapy. The figure depicts another mechanism of action of targeted drug activation with prodrug nanocarrier and non-encapsulated enzyme system. The enzyme is PEGylated which makes it more stable, has longer plasma half-time, and prevents or reduces immune response to the enzyme if it is of heterogenic origin. Both the nanocarrier and PEGylated enzyme will accumulate in tumor through the mechanism of EPR and killing of the tumor cells occurs. The mechanism for non-toxicity to RES is as described for FIG. 1.

FIG. 3. Prodrug nanocarrier and enzyme expression vector system for targeted cancer therapy. The figure depicts yet another mechanism of action of targeted drug activation with a different duel nanocarrier system. Like the inventive concepts disclosed in above figures, the prodrug is encapsulated in nanocarrier. However, the activating enzyme is synthesized by tumor cells or non-tumoral cells in the tumor. These cells are transduced by an enzyme expression construct carried in a different nanocarrier (an expression vector). Because the expression construct contains a signaling sequence that directs extracellular secretion or membrane expression of the enzyme, the synthesized enzyme is accessible to the prodrug. Also, by attachment of tumor targeting moiety to the vector and/or by cloning a tumor specific promoter in the expression construct, such as promoter for MMP (matrix metalloproteinases), HIF (hypoxia-inducible factor) and VEGF (vascular endothelial growth factor), or VEGF receptor, the enzyme will be mainly expressed in the tumor. As a result, activation of prodrug will occur mainly in tumor. Other features can also be designed into the nanocarrier, such as the structure which facilitates the escape of expression construct from endosomal-lysosomal vesicles into the cytoplasm and nucleus of cells.

### DETAILED DESCRIPTION OF EXEMPLARY EMBODIMENTS

The presently disclosed and claimed inventive concepts relates to methods of delivery of active pharmaceutical agent to the site of interest such as tumor and site of inflammation. Such methods have no or less toxicity to reticuloendothelial system and other extratumoral or extra-inflammatory organs or tissues than previously described methods. While the description sets forth various embodiment specific details, it will be appreciated that the description is illustrative only and should not be construed in any way as limiting the invention. Furthermore, various applications of the inventive concepts and modification thereof, which may occur to those who are skilled in the art, are also encompassed by the general concepts described below. Also, it is to be understood that the phraseology and terminology employed herein is for the purpose of description and should not be regarded as limiting.

It must be noted that, as used in this embodiment and the appended claims, the singular forms “a”, “an”, and “the” includes plural referents unless the context clearly dictates otherwise. Likewise, the plural terms shall also include the singularity unless otherwise required by the context.

In describing and claiming the present invention, the following terminology will be used in accordance with the definitions set out below.

As used herein, “comprising”, “including”, “containing”, and grammatical equivalents thereof are inclusive or open-ended terms that do not exclude additional, unrecited elements or method steps.

The use of the term “or” in the claims is used to mean “and/or” unless explicitly indicated to refer to alternatives only or the alternatives are mutually exclusive, although the disclosure supports a definition that refers to only alternatives and “and/or”.

As used herein, “PEGylation” or “PEGylated” means covalently coupling of or coupled with poly(ethyleneglycol) (PEG). “Expression construct” refers to DNA sequences which include gene and genetic control elements that allow the expression of the gene in a selected recombinant host. The term “gene” is used for simplicity to refer to a functional protein, polypeptide, or peptide encoding unit. “Transduction” or “transduced” means transfer of or transferred with genetic material when human and animal cells are concerned. The genetic material can be plasmid, a process named transfection, or viral vector, a process named infection. “Promoter” refers to, in the realm of genetics, a region of DNA that facilitates the transcription of the gene it regulates. “Nanocarrier” designates a nano-sized structure, ranging from 10 to 500 nm in diameter, that carries pharmaceutical or therapeutic agent to the location of interest inside human or animal body. “Amphiphilic block copolymer” refers to a polymer with two or more homopolymer subunits linked by covalent bonds and contains at least one hydrophilic and one hydrophobic subunit or block.

### EXAMPLE 1

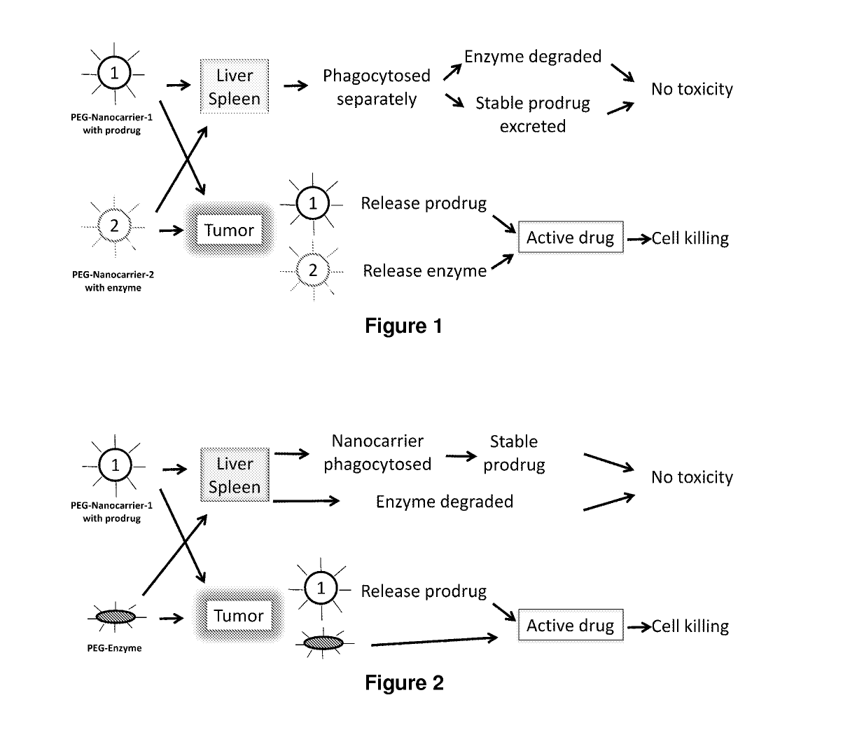

In one embodiment of FIG. 1, the two-step delivery system is composed of PEGylated polymeric micelles and PEGylated liposomes. The micelles are composed of amphiphilic block copolymer with PEG as the hydrophilic block, and encapsulate cephalosporin-camptothecin prodrug in its hydrophobic core. The liposomes are composed of PEG-lipids, and encapsulate beta-lactamase. Both nanocarriers are administered into animal or human patient intravenously. Liver and spleen are used as the exemplary model of reticuloendothelial system (RES). Traveling inside the blood circulation, both nanocarriers are either engulfed by RES or pass through. When engulfed, the prodrugs are not activated inside the lysosomes, and the enzymes are denatured or degraded. Therefore, no toxicity occurs. Contrarily, through the effect of EPR, both nanocarriers accumulate in the tumor and encapsulated cargo is released. Camptothecin is freed from cephalosporin-camptothecin prodrug by the action of beta-lactamase in the tumor interstitial space and exerts therapeutic effect locally.

### EXAMPLE 2

In an additional embodiment of FIG. 1, both nanocarriers are linked on the free end of PEG chain with targeting moiety, such as antibody, for tumor, tumor extracellular component, or tumor vasculature. The active targeting will increase the accumulation of both nanocarriers in the tumor and, therefore, increase the therapeutic efficacy. Similar to what is described in Example 1, the targeting moiety linked nanocarriers will not cause toxicity to RES.

### EXAMPLE 3

In a further embodiment of FIG. 1, the nanocarriers are made with block copolymers that undergo hydrolysis or structural changes in acidic environment. Most solid tumors are acidic in their interstitial space. Therefore, when the nanocarriers are accumulated in these tumors, accelerated release of prodrugs and enzymes occur and better therapeutic efficacy will be achieved.

### EXAMPLE 4

In one embodiment of FIG. 2, the PEGylated polymeric micelles which encapsulate cephalosporin-camptothecin prodrug, and PEGylated beta-lactamase are administered respectively into animal or human patients. PEGylation of beta-lactamase reduces or eliminates the immune response to the enzyme. It also makes the enzyme more stable, having longer plasma half-time, and increasing accumulation in tumor. Because the prodrug is stable in the lysosomal system, no toxicity occurs to RES organs when the micelle is engulfed by RES. If the PEGylated beta-lactamase is taken up by RES, it is denatured and degraded inside the endosome and lysosome, and unable to activate the prodrugs. When the micelles and PEGylated lactamase accumulate in tumor through the effect of EPR, the released prodrug is activated and killing of the tumor cells occurs locally.

### EXAMPLE 5

In another embodiment of FIG. 2, the PEGylated beta-lactamase is linked on the free end of PEG chain with targeting moiety, such as antibody, for tumor, tumor extracellular component, or tumor vasculature. The prodrug encapsulated micelles can be linked with the targeting moiety or not linked. The active targeting through targeting moiety will increase the accumulation of the enzyme and micelles in the tumor and, therefore, increase the therapeutic efficacy.

### EXAMPLE 6

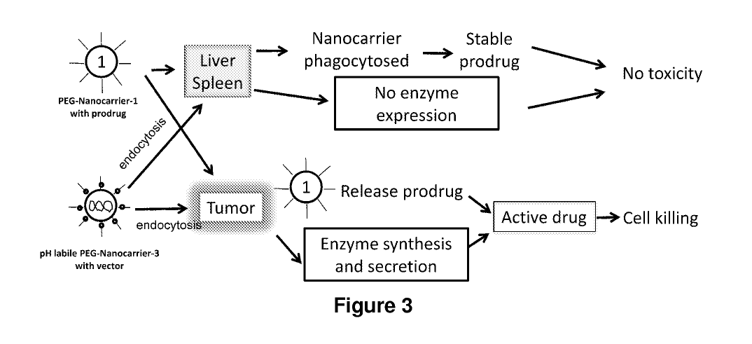

In one embodiment of FIG. 3, the cephalosporin-camptothecin prodrug encapsulating nanocarrier is PEGylated polymeric micelle and beta-lactamase is produced by tumor cells or non-tumoral cells in the neighborhood of the tumor cells. The transduction of these cells takes place when the cells endocytose the polymeric micelles carrying beta-lactamase expression construct. The gene delivery micelles are made of PEG-polycation copolymer where the negatively charged gene is complexed with cationic block of the copolymer. The expression construct contains signaling sequence that directs extracellular secretion or cell membrane expression of the enzyme. It also contains tumor specific promoter, such as the promoter for MMP (matrix metalloproteinases), HIF (hypoxia-inducible factor), VEGF (vascular endothelial growth factor), or VEGF receptor. As is known to one ordinary skilled in the art, MMP, HIF, VEGF or VEGFR is over-expressed for most invasive cancer cells. The designs of the micelle, tumor targeting moiety and tumor expression promoter, make sure that beta-lactamase is produced mainly inside the tumor. In addition, since prodrugs are encapsulated inside the nanocarrier, any residual expression of the enzyme by the cells outside of tumor will not result in substantial prodrug activation and toxicity.

U.S. Pat. No. 7,304,045B2 titled Nanoparticles For Targeting Hepatoma Cells, described a dual-particle tumor targeting system (Sung et al. 2007). In this system, one type of nanoparticle which encapsulates prodrug is conjugated with targeting ligand, and the other nanoparticle which encapsulates enzyme expression gene is not conjugated with targeting ligand. The enzyme is expressed inside the cells after the nanoparticle is taken up. When the same cell takes up the first nanoparticle through binding of the targeting ligand to the receptor of the cell, the encapsulated prodrug is released and activated inside the cell by the enzyme and the cell is killed as a result.

The embodiment of this invention does not require taking up two different nanocarriers by the same cells. In addition, the expressed enzyme is delivered to the outside of the cells and the activated drug can kill the cell whether it expresses the gene or not.

### EXAMPLE 7

In an alternative embodiment of FIG. 3, beta-lactamase is produced by autologous or allogenic multipotent mesenchymal stromal cells (MSC). These cells have the ability to home to tumor (Bexell et al. 2010; Harrington et al. 2002; Nakashima et al. 2010; Goldstein et al. 2010). The cells are transduced in vitro by the enzyme expression construct with the signaling sequence that directs extracellular secretion or cell membrane expression of the enzyme, before delivered back into the cancer patient. The engineered cells migrate and home to tumor where they synthesize and secrete prodrug activating enzyme. As a result, the released prodrug is activated in the interstitial space of the tumor and kills the tumor cells with little side effect outside the tumor.

### EXAMPLE 8

In a different embodiment, the prodrug encapsulated in PEGylated polymeric micelles is cephalosporin-dexamethasone. Because of leaky vasculature in the inflammation sites of the affected joints in rheumatoid arthritis patient, the micelle is accumulated in these sites and releases cephalosporin-dexamethasone prodrug which is activated by PEGylated beta-lactamase also accumulated at the site. Therefore, dexamethasone can be used for longer term to treat rheumatoid arthritis without much side-effect.

Although the present invention is described with reference to specific details of certain embodiments, it is not intended that such details should be regarded as limitations upon the scope of the invention except as and to the extent that they are in the accompanying claims. Many modifications and variations are possible in light of the above disclosure.

### REFERENCE LIST

Albert B., Johnson A, Lewis J, Raff M, Roberts K, Walter P (2002) ‘Molecular Biology of the Cell, 4th edition.’ (Garland Science: New York)

Alexis F, Pridgen E, Molnar L K, Farokhzad O C (2008) Factors affecting the clearance and biodistribution of polymeric nanoparticles. Mol. Pharm. 5, 505-515.

Alexis F, Pridgen E M, Langer R, Farokhzad O C (2010) Nanoparticle technologies for cancer therapy. Handb. Exp. Pharmacol. 55-86.

Allen T M, Martin F J (2004) Advantages of liposomal delivery systems for anthracyclines. Semin. Oncol. 31, 5-15.

Alley S C, Okeley N M, Senter P D (2010) Antibody-drug conjugates: targeted drug delivery for cancer. Curr Opin. Chem. Biol. 14, 529-537.

Altaner C (2008) Prodrug cancer gene therapy. Cancer Lett. 270, 191-201.

Bagshawe K D (2009) Targeting: the ADEPT story so far. Curr. Drug Targets. 10, 152-157.

Bexell D, Scheding S, Bengzon J (2010) Toward brain tumor gene therapy using multipotent mesenchymal stromal cell vectors. Mol. Ther. 18, 1067-1075.

Cui J, Li C, Guo W, Li Y, Wang C, Zhang L, Zhang L, Hao Y, Wang Y (2007) Direct comparison of two pegylated liposomal doxorubicin formulations: is AUC predictive for toxicity and efficacy? J. Control Release 118, 204-215.

Duncan R, Gac-Breton S, Keane R, Musila R, Sat Y N, Satchi R, Searle F (2001) Polymer-drug conjugates, PDEPT and PELT: basic principles for design and transfer from the laboratory to clinic. J. Control Release 74, 135-146.

Fang J, Sawa T, Akaike T, Maeda H (2002) Tumor-targeted delivery of polyethylene glycol-conjugated D-amino acid oxidase for antitumor therapy via enzymatic generation of hydrogen peroxide. Cancer Res. 62, 3138-3143.

Gerber H P, Senter P D, Grewal I S (2009b) Antibody drug-conjugates targeting the tumor vasculature: Current and future developments. MAbs. 1, 247-253.

Gerber H P, Senter P D, Grewal I S (2009a) Antibody drug-conjugates targeting the tumor vasculature: Current and future developments. MAbs. 1, 247-253.

Goldstein R H, Reagan M R, Anderson K, Kaplan D L, Rosenblatt M (2010) Human Bone Marrow-Derived MSCs Can Home to Orthotopic Breast Cancer Tumors and Promote Bone Metastasis. Cancer Res. 70, 10044-10050.

Greish K (2007a) Enhanced permeability and retention of macromolecular drugs in solid tumors: a royal gate for targeted anticancer nanomedicines. J. Drug Target 15, 457-464.

Greish K (2007b) Enhanced permeability and retention of macromolecular drugs in solid tumors: a royal gate for targeted anticancer nanomedicines. J. Drug Target 15, 457-464.

Harrington K, varez-Vallina L, Crittenden M, Gough M, Chong H, Diaz R M, Vassaux G, Lemoine N, Vile R (2002) Cells as vehicles for cancer gene therapy: the missing link between targeted vectors and systemic delivery? Hum. Gene Ther. 13, 1263-1280.

Houston T A (2007) Painting the target around the arrow: two-step prodrug therapies from a carbohydrate chemist's perspective. Curr. Drug Deliv. 4, 264-268.

Huang X, Peng X, Wang Y, Wang Y, Shin D M, EI-Sayed MA, Nie S (2010) A reexamination of active and passive tumor targeting by using rod-shaped gold nanocrystals and covalently conjugated peptide ligands. ACS Nano. 4, 5887-5896.

Hume D A (2006) The mononuclear phagocyte system. Curr Opin. Immunol. 18, 49-53.

Huober J, Fett W, Nusch A, Neise M, Schmidt M, Wischnik A, Gerhardt S, Goehler T, Luck H J, Rost A (2010) A multicentric observational trial of pegylated liposomal doxorubicin for metastatic breast cancer. BMC.Cancer 10, 2.

Kim S, Kim J H, Jeon O, Kwon I C, Park K (2009) Engineered polymers for advanced drug delivery. Eur. J. Pharm. Biopharm. 71, 420-430.

Kratz F, Muller I A, Ryppa C, Warnecke A (2008) Prodrug strategies in anticancer chemotherapy. Chem Med Chem. 3, 20-53.

Low P S, Kularatne S A (2009) Folate-targeted therapeutic and imaging agents for cancer. Curr Opin. Chem. Biol. 13, 256-262.

Maeda H, Bharate G Y, Daruwalla J (2009) Polymeric drugs for efficient tumor-targeted drug delivery based on EPR-effect. Eur. J. Pharm. Biopharm. 71, 409-419.

Matsumura Y, Kataoka K (2009) Preclinical and clinical studies of anticancer agent-incorporating polymer micelles. Cancer Sci. 100, 572-579.

Mikhail A S, Allen C (2009) Block copolymer micelles for delivery of cancer therapy: transport at the whole body, tissue and cellular levels. J. Control Release 138, 214-223.

Nakashima H, Kaur B, Chiocca E A (2010) Directing systemic oncolytic viral delivery to tumors via carrier cells. Cytokine Growth Factor Rev. 21, 119-126.

O'Brien M E (2008b) Single-agent treatment with pegylated liposomal doxorubicin for metastatic breast cancer. Anticancer Drugs 19, 1-7.

O'Brien M E (2008a) Single-agent treatment with pegylated liposomal doxorubicin for metastatic breast cancer. Anticancer Drugs 19, 1-7.

Osada K, Christie R J, Kataoka K (2009) Polymeric micelles from poly(ethylene glycol)-poly(amino acid) block copolymer for drug and gene delivery. J. R. Soc.Interface 6 Suppl 3, S325-S339.

Pasut G, Veronese F M (2009) PEG conjugates in clinical development or use as anticancer agents: an overview. Adv. Drug Deliv. Rev. 61, 1177-1188.

Roseng L, Tolleshaug H, Berg T (1992) Uptake, intracellular transport, and degradation of polyethylene glycol-modified asialofetuin in hepatocytes. J. Biol. Chem. 267, 22987-22993.

Satchi-Fainaro R, Hailu H, Davies J W, Summerford C, Duncan R (2003) PDEPT: polymer-directed enzyme prodrug therapy. 2. HPMA copolymer-beta-lactamase and HPMA copolymer-C-Dox as a model combination. Bioconjug. Chem. 14, 797-804.

Sawa T, Wu J, Akaike T, Maeda H (2000) Tumor-targeting chemotherapy by a xanthine oxidase-polymer conjugate that generates oxygen-free radicals in tumor tissue. Cancer Res. 60, 666-671.

Singh Y, Palombo M, Sinko P J (2008) Recent trends in targeted anticancer prodrug and conjugate design. Curr. Med. Chem. 15, 1802-1826.

Sung, H W, Hsu, H K, and Tu H. Nanoparticles for targeting hepatoma cells. [U.S. Pat. No. 7,304,045B2]. 2007. Ref Type: Patent

Teicher B A (2009) Antibody-drug conjugate targets. Curr. Cancer Drug Targets. 9, 982-1004.

Torchilin V (2009) Multifunctional and stimuli-sensitive pharmaceutical nanocarriers. Eur. J. Pharm. Biopharm. 71, 431-444.

Torchilin V P (2010) Passive and active drug targeting: drug delivery to tumors as an example. Handb. Exp. Pharmacol. 3-53.

Veronese F M, Mero A (2008) The impact of PEGylation on biological therapies. BioDrugs. 22, 315-329.

Wang X, Wang Y, Chen Z G, Shin D M (2009) Advances of cancer therapy by nanotechnology. Cancer Res. Treat. 41, 1-11.

I claim:

1. A two-step targeted tumor therapy method comprising nanocarrier with prodrug encapsulated thereto, and enzyme to activate said prodrug, whereby said nanocarrier and enzyme are suspended in a solution for delivery into patient simultaneously or at different time points, and said prodrug released from said nanocarrier is activated by said enzyme mainly in the interstitial space of tumor.

2. The method of claim 1, wherein said nanocarrier is selected from a group of nano-sized structures that are able to carry the prodrugs to the locations of interest inside the patient, and protect the prodrugs from activation before their release.

3. The method of claim 2, wherein said nano-sized structure is a nanoparticle that is composed of amphiphilic block copolymers with a hydrophobic inner core and hydrophilic outer shell, such as polymeric micelle and polymeric nanoparticle.

4. The nanoparticle of claim 3, wherein said hydrophilic outer shell is poly(ethyleneglycol) (PEG), or other molecule that enhances the stability and circulation time, and reduces clearance and immune response of said nanoparticle.

5. The method of claim 2, wherein said nano-sized structure alternatively takes the form of a vesicle with the hydrophilic groups facing outside and the interior space of said vesicle, and is selected from a group including nanovesicle, liposome, and polymersome.

6. The vesicle of claim 5, wherein said out-facing hydrophilic group is poly(ethyleneglycol) (PEG), or other molecule that enhances the stability and circulation time, and reduces clearance and immune response of said vesicle.

7. The method of claim 1, wherein said prodrug is the hydrophobic block of an amphiphilic block copolymer of which the nanocarrier is composed.

8. The method of claim 1, wherein said nanocarrier further comprises a targeting moiety joined thereto for targets in tumor cells, tumor extracellular component, or tumor vasculature.

9. The nanocarrier of claim 8, wherein said targeting moiety includes antibody, antibody fragment, antibody mimetic, aptamer, vitamin, sugar moiety, integrin ligand, any other cell surface receptor ligand, and extracellular component ligand.

10. The method of claim 1, wherein said nanocarrier additionally contains enzyme degradable components and/or tumor extracellular microenvironment, such as pH, temperature, or oxygen level, responsive structures, that control the stability of said nanocarrier and regulate the release of prodrug carried by said nanocarrier.

11. The method of claim 1, wherein said enzyme is a prodrug activating enzyme, either of eukaryotic or prokaryotic origin, or a non-enzyme prodrug activating agent.

12. The method of claim 1, wherein said enzyme is not encapsulated in a nanocarrier.

13. The method of claim 1, wherein said enzyme is alternatively carried in a nanocarrier, being encapsulated in the core of said nanocarrier or associated with said nanocarrier.

14. The nanocarrier of claim 13, further possessing the same characteristics as for prodrug encapsulating nanocarrier stated in claims 2, 3, 4, 5, 6, 8, 9, and 10.

15. The enzyme of claim 12, further being PEGylated or conjugated with any other molecule that makes said enzyme more soluble, more stable, less degradable, staying in blood circulation for longer time, and minimally or less immunogenic, when compared with non-conjugated enzyme.

16. The PEGylated enzyme or any other molecule conjugated enzyme of claim 15, further being conjugated on the free end of PEG chain or said other molecule with targeting moiety for tumor cells, tumor extracellular component, or tumor vasculature.

17. The enzyme of claim 16, wherein said targeting moiety includes antibody, antibody fragment, antibody mimetic, aptamer, vitamin, sugar moiety, integrin ligand, any other cell surface receptor ligand, and extracellular component ligand.

18. The method of claim 1, wherein said enzyme is directly conjugated with targeting moiety for tumor cells, tumor extracellular component, or tumor vasculature.

19. The enzyme of claim 18, wherein said targeting moiety includes antibody, antibody fragment, antibody mimetic, aptamer, vitamin, sugar moiety, integrin ligand, any other cell surface receptor ligand, and extracellular component ligand.

20. The method of claim 1, wherein said nanocarrier and enzyme are delivered into patient intravenously, intra-arterially, intraperitoneally, intracerebrally, intracerebroventricularly, intratracheally, or intratumorally, depending on the type of tumor and the stage of the disease.

21. The method of claim 1, wherein said prodrug is minimally toxic or much less toxic than the parent drug.

22. The method of claim 1, wherein said prodrug is not activated in lysosome by lysosomal enzymes, or acidic environment, but only by the prodrug activating enzyme or a non-enzyme prodrug activating agent.

23. The method of claim 1, wherein said prodrug is a precursor or derivative form of a pharmaceutically active substance, either hydrophilic or hydrophobic, which includes but not limited to cytotoxic or anti-cancer therapeutic agent such as doxorubicin, etoposide, cytarabine, cisplatin, taxanes, vinca alkaloids, camptothecin, gemcitabine, 5-FU, HDAC inhibitors, and proteasome inhibitors.

24. The method of claim 1, wherein said tumor is any type of solid tumor or cancer, including lung cancer, breast cancer, liver cancer, pancreatic cancer, prostate cancer, colon cancer, renal cancer, head and neck cancer, esophageal cancer, stomach cancer, and brain tumors.

25. A different two-step targeted tumor therapy method comprising nanocarrier with prodrug encapsulated thereto, and enzyme produced inside said tumor.

26. The method of claim 25, wherein said enzyme is produced by tumor cells or non-tumoral cells in the neighborhood of said tumor cells inside said tumor.

27. The method of claim 26, wherein said tumor cells or non-tumoral cells are transduced by gene which contains the enzyme expression construct with signaling sequence that directs extracellular secretion or cell membrane expression of said enzyme.

28. The enzyme expression construct of claim 27, further containing tumor specific promoter that regulates the expression of said enzyme in said tumor, including but not limited to promoters of MMP (matrix metalloproteinases), HIF (hypoxia-inducible factor), VEGF (vascular endothelial growth factor), and VEGF receptor.

29. The method of claim 27, wherein said gene is carried to tumor by a nanocarrier or other vector which is capable of accumulating in said tumor or is targeted for targets in tumor cells, tumor extracellular component, or tumor vasculature.

30. The method of claim 25, wherein said enzyme is alternatively produced by autologous or allogenic human cells which are transduced in vitro by the enzyme expression construct before transferred back into the cancer patient; said expression construct containing signaling sequence that directs extracellular secretion or cell membrane expression of said enzyme.

31. The method of claim 30, wherein said human cells have the ability to home to tumor, including, but not limited to, mesenchymal stromal cells, neural stem cells, endothelial cells, hematopoietic cells, skin derived cells, and endometrial precursor cells.

32. The method of claim 25, wherein said tumor is any type of solid tumor or cancer, including lung cancer, breast cancer, liver cancer, pancreatic cancer, prostate cancer, colon cancer, renal cancer, head and neck cancer, esophageal cancer, stomach cancer, and brain tumors.

33. Another different two-step targeted therapeutic method comprising nanocarrier with prodrug encapsulated thereto and enzyme for treatment of inflammatory diseases such as rheumatoid arthritis, inflammatory bowel disease, lupus erythematosus, and atherosclerosis, whereby said prodrug released from said nanocarrier is activated by said enzyme mainly in the inflammation sites of the inflammatory diseases.

34. The method of claim 33, wherein said nanocarrier and enzyme are targeted for inflammation sites, and said prodrug is derived from anti-inflammatory drugs or agents, including steroids, methotrexate, azathioprine, and ciclosporin.
